(19) United States
(12) Patent Application Publication
(10) Pub. No.: US 2012/0116506 A1
(43) Pub. Date: May 10, 2012

(54) ACCOMMODATING INTRAOCULAR LENS SYSTEM INCLUDING A BAG

(76) Inventor: David C. Compertore, Penfield, NY (US)

(21) Appl. No.: 12/942,105

(22) Filed: Nov. 9, 2010

(51) Int. Cl. A61F 2/16

(52) U.S. Cl. 623/6.37

(57) ABSTRACT

An accommodating intraocular lens kit for implantation into an eye, comprising an accommodating intraocular lens having at least one biasing element, a biocompatible bag having a size and shape to conform to an interior surface of a capsular bag, and an apparatus for transferring accommodative force of the eye from the eye to the at least one biasing element. The apparatus may comprise a transfer rod or a magnetic medium.

### FIELD OF INVENTION

The present invention relates to accommodating intraocular lens systems.

### BACKGROUND OF THE INVENTION

FIG. 1 illustrates a cross-sectional view of a human eye 10 having an anterior chamber 12 and a posterior chamber 14 separated by an iris 30. Within the posterior chamber 14 is a capsular bag 16 which holds the eye's natural crystalline lens 17. Light enters the eye by passing through cornea 18. The cornea and crystalline lens act together to direct and focus the light onto retina 20. The retina is connected to optic nerve 22 which transmits images received by the retina to the brain for interpretation. Eye 10 has a visual axis VA.

In response to the sharpness of the image received by the retina, the brain operates to contract or relax ciliary muscle 26. Ciliary muscle 26 is disposed within ciliary body 28, and upon contraction of the ciliary muscle, the ciliary body is caused to move. To achieve near-focus accommodation, the ciliary muscle is contracted thereby causing the ciliary body to relax tension on zonules 27 which permits the capsular bag and lens 17 to become more rounded. To achieve far focus (i.e., disaccommodation), the ciliary muscle is relaxed thereby increasing tension on zonules 27 which causes the capsular bag and lens 17 to become flatter.

In an eye where the natural crystalline lens has been damaged (e.g., clouded by cataracts), the natural lens is no longer able to properly focus and/or direct incoming light to the retina. As a result images become blurred. A well known surgical technique to remedy this situation involves removal of a damaged crystalline lens through a hole in the capsular bag known as a capsularhexis (also referred to simply as a rhexis). Subsequently, an artificial lens known as an intraocular lens (IOL) can be placed into the evacuated capsular bag through the rhexis.

Conventional IOLs are typically fixed-focus lenses. Such lenses are usually selected to have a power such that the patient has a fixed focus for distance vision, and the patient requires spectacles or contact lenses to permit near vision. In recent years extensive research has been carried out to develop IOLs having variable focus capability. Such IOLs are known as accommodating IOLs (AIOLS). The term “AIOLs” refers to, lenses having a single optical element and lenses having multi optical elements, and lenses that rely on a change of shape and/or accommodative translational movement.

AIOLs permit a wearer to have accommodative vision. AIOLs are typically located in the posterior chamber (e.g., in the capsular bag) and are designed to provide variable focal power in accordance with contraction and relaxation of the ciliary muscle and corresponding tension or a lack of tension exerted on the capsular bag 16.

Problems with AIOLs that have been implanted to date include that (1) they have provided less than desirable amounts of accommodation, and (2) they have acted unpredictably when implanted in an eye.

### SUMMARY

Aspects of the present invention are directed to an accommodating intraocular lens kit for implantation into an eye, comprising an accommodating intraocular lens having at least one biasing element, a biocompatible bag having a size and shape to conform to an interior surface of a capsular bag, and a means for transferring accommodative force of the eye from the eye to the at least one biasing element.

The accommodating intraocular lens may comprise a first optical element and a second optical element. In other embodiments, the accommodating intraocular lens consists of a single optical element.

In some embodiments, the bag has a spring constant of at least 0.5 mN/mm. In some embodiments, the bag has a spring constant of at least 1.0 mN/mm. In some embodiments, the bag has a spring constant less than 200 mN/mm. The bag may have a hole sized and shaped to permit the accommodating intraocular lens to fit therethrough.

In some embodiments, the means for transferring comprises at least two transfer rods. The transfer rods may each have a length selected to permit coupling between the biasing elements and the zonules or ciliary body of the eye. For example, the transfer rods each have a length between 2-7 mm.

In some embodiments, the means for transferring comprises at least one magnetic medium connected to the biasing element and at least one magnetic medium adapted to be connected to the zonules or ciliary body.

Another aspect of the invention is directed to a method of implantation of an accommodative lens in an eye, comprising inserting a biocompatible bag into an eye, inserting an accommodating intraocular lens having at least one biasing element into the biocompatible bag after the biocompatible bag is in the eye, and connecting a means for transferring accommodative force between the zonules or ciliary body of the eye, and the at least one biasing element.

The step of connecting the means for transferring may comprise connecting a transfer rod between the biasing element and the zonules or the ciliary body of the eye.

The step of connecting the means for transferring may comprise connecting at least one magnetic medium to the at least one biasing element and connecting at least one magnetic medium to the zonules or ciliary body.

### BRIEF DESCRIPTION OF THE DRAWINGS

Illustrative, non-limiting embodiments of the present invention will be described by way of example with reference to the accompanying drawings, in which the same reference number is used to designate the same or similar components in different figures, and in which:

FIG. 1 is a schematic illustration of a cross-sectional view of a human eye;

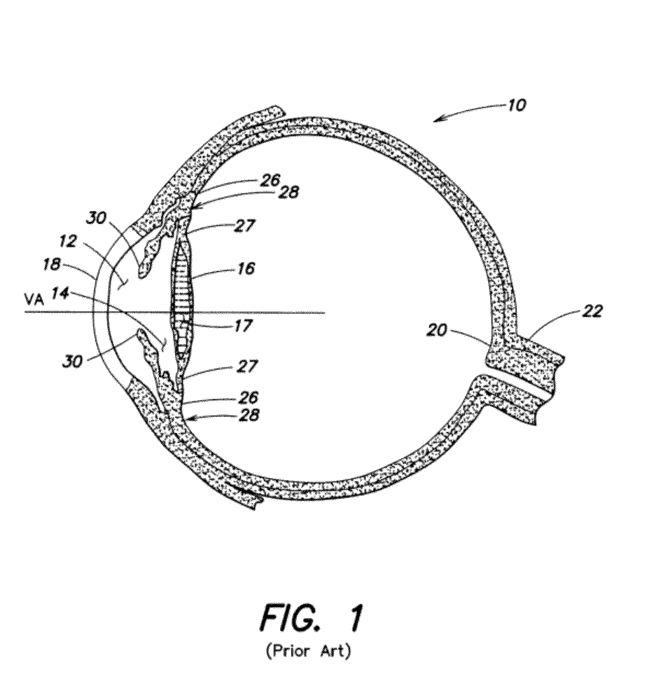

FIG. 2A is a cross-sectional side view of an embodiment of an accommodating intraocular lens kit according to aspects of the present invention, the lens shown in an assembled state within an eye;

FIG. 2B is a schematic drawing of components of the AIOL kit of FIG. 2A in an unassembled state; and

FIGS. 3A and 3B are cross-sectional side views of another embodiment of an accommodating intraocular lens kit for use in embodiments according to aspects of the present invention.

### DETAILED DESCRIPTION

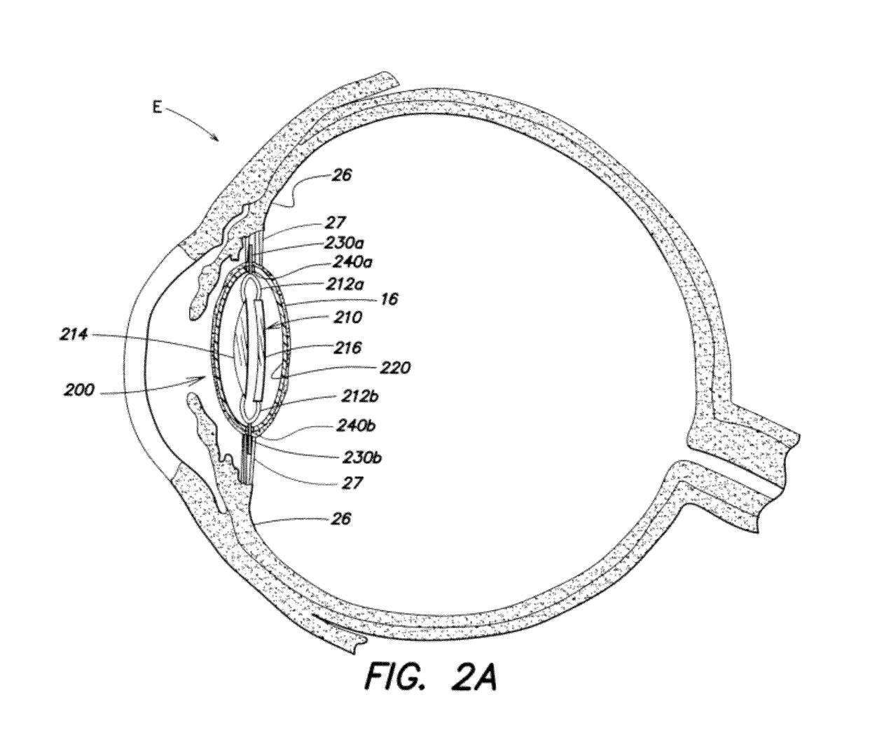

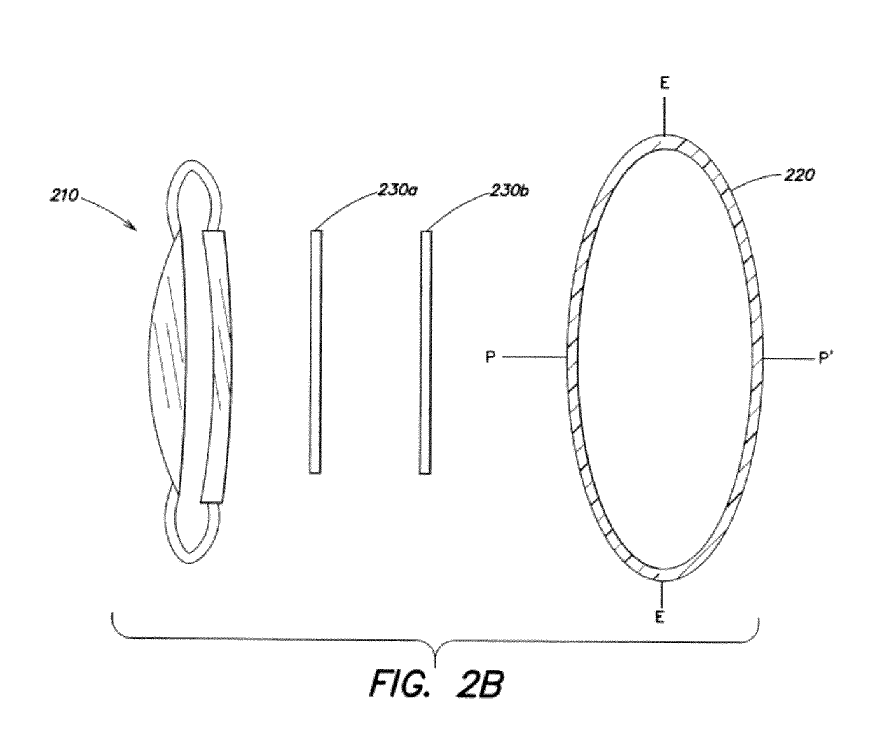

FIG. 2A is a cross-sectional side view of an embodiment of an accommodating intraocular lens kit 200 according to aspects of the present invention, the kit being in an assembled state within an eye E. FIG. 2B is a schematic drawing of components of the AIOL kit of FIG. 2A in an unassembled state. The kit comprises an accommodating lens 210 comprising two optical elements 214 and 216, a biocompatible bag 220 and transfer rods 230a and 230b. It will be appreciated that components of a kit may be provided to a surgical staff in an unassembled form, for example, in a manner as discussed below, or provided in a partially or a fully assembled state.

Accommodating intraocular lens 210 comprises two biasing elements 212a and 212b. However, embodiments of lenses for use kits according to the present invention comprise at least one biasing element. For example, accommodating intraocular lenses may have three biasing element and be configured such as lenses described in U.S. Pat. No. 6,488,708 to Sarfarazi. In addition to positioning lenses to achieve a focused state, biasing elements may operate as haptics and provide centration of the lens within the capsular bag.

Although the illustrated AIOL embodiment comprises two optical elements, it will be appreciated that an accommodating lens may comprise a single element or three or more elements. An example of a single optical element AIOL is given in U.S. Pat. No. 5,674,282 to Cumming.

Biocompatible bag 220 has a size and shape to conform to an interior surface of a capsular bag 16. The bag has poles P and P′ (which are designed to substantially align with an eye's visual axis) and an equator E. Typically the biocompatible bag will have a shape where the cross sections are oval (e.g., an ovoid or ellipsoid shape), the equatorial diameter is in the range 10-13 mm, and the distance between the poles is 3-5 mm. Bag 220 is dimensioned to have a rigidity selected to keep the capsular bag from collapsing onto lens 210 so as to avoid interference of the capsular bag with the accommodative movement of the AIOL. It will be appreciated that the rigidity needed to achieve such a result varies among individuals and, for a given individual, varies with age. Rigidity, as the term is used herein, can be expressed as the bag's spring constant, when the bag is stretched by substantially point contacts at its poles. According to aspects of the present invention, a bag 220 has a spring constant of at least 0.5 mN/mm and, in some embodiments at least 1.0 mN/mm.

It will be appreciated that, although a bag of greater rigidity may be capable of keeping the capsular bag from collapsing onto the lens, it is also desirable that the bag be easily inserted through a relatively small incision in the cornea and capsular bag of the eye. Accordingly, a spring constant of less than 200 mN/mm is generally desirable, and in some embodiments less than 15 mN/mm. For example, a bag may comprise a silicone material.

A biocompatible bag, prior to being inserted into an eye, may have one or more holes through which an accommodating intraocular lens is inserted after the bag has been inserted into the capsular bag. Alternatively, the bag may be inserted into the capsular bag without a hole for insertion of the AIOL, and one or more holes can be subsequently formed in the bag for insertion of the AIOL and any subsequent assembly or manipulation of the intraocular lens as described below.

It will be appreciated that any holes in the bag that are either prior-formed or formed in situ can have a standard size and shape as would be conventionally made in a capsular bag for insertion of the AIOL or another size and/or shape to facilitate insertion of a lens or other components of a kit. It will be appreciated that, whereas the capsular bag may have a rhexis formed in it for removal of the crystalline lens, a bag may be placed in the capsular bag which covers at least a portion of said capsularhexis and may therefore serve to ameliorate any damage done to the capsular bag during the formation of the capsularhexis. Additionally, because the bag covers the interior of the capsular bag and prevents contact of the intraocular lens with the inner surface of the capsular bag, the bag may serve to prevent posterior capsular opacification (PCO).

Transfer rods 230a and 230b have a length that is selected to permit connection between one of said biasing members 212a and 212b and zonules 26 which extend through bag 220. As the ciliary muscle 26 relaxes to achieve distance vision, the transfer rods exert a radially outward force on the biasing elements, thereby drawing lenses 214 and 216 together; and as the ciliary muscle contracts to achieve near vision, the transfer rods exert a radially inward force on biasing elements 212a and 212b, thereby causing lenses 214 and 216 to move apart from one another. It will be appreciated that the transfer rods thereby operate as a means for transferring accommodative force of said eye from the eye to an at least one biasing element 212a and 212b. Transfer rods are typically made of a material that is more rigid than the material used to make the lens. For example, if the lenses are made of silicone, the transfer rods may be made of PMMA. Although connection of the transfer rods to the zonules may be advantageous in some instances, in other instances connection with, or contact with out connection to the ciliary body may be desirable. Each transfer rod may have a length in the range 2-7 mm. It should be appreciated that, although the embodiments described herein cause optical elements to achieve their accommodative state upon contraction of the ciliary muscle and their disaccommodative state upon relaxation of the ciliary muscle, a lens within the scope of aspects of the present invention could (e.g., with appropriate levering) achieve its disaccommodative state upon contraction of the ciliary muscle and its accommodative state upon relaxation of the ciliary muscle.

To attach the transfer rods to the lens, any suitable attachment technique may be used which is capable of facilitating transfer of a force from the zonules or the ciliary body to the biasing elements to move and/or deform either or both of optical element 214 and 216 (in the case of a dual-optical element lens) and appropriately move and/or deform an optical element (in the case of a single optical element lens). For example, an adhesive, a clip, a pin, snap or suturing may be used to achieve the attachment. To attach the transfer rods to the zonules, the rod may be entwined in the zonules, sutured thereto or connected by any other suitable attachment technique. A rod extending to the ciliary body may be attached or connected thereto or simply rest against the ciliary body.

Set forth below are techniques for implantation of an AIOL kit according to aspects of the present invention; however, other techniques are possible, and apparatus as described herein are not limited to any technique of implantation. To implant the components of a kit according to this embodiment, a patient's crystalline lens is typically first removed. Crystalline lens removal can be achieved using any suitable technique (e.g., formation of a capsularhexis and emulsification using ultrasound and/or laser energy). After the lens is removed, the bag can be inserted into the eye through the capsularhexis or another port in the eye. The biocompatible bag can be inserted using forceps or a syringe-like injector. The size of the port and/or injector that may be used to insert the bag is, at least in part, determined by the rigidity of the biocompatible bag. After the bag is in place within the capsular bag, the accommodating lens can be inserted into the biocompatible bag using forceps or a syringe-like injector. As indicated above, insertion of the accommodating lens can be accomplished through a hole in the bag that is preformed or through a subsequently formed hole. It will be appreciated that a hole in the capsular bag that is aligned with or otherwise in communication with the hole in the biocompatible bag may be required to permit placement of the accommodating lens in the bag. The IOL may be injected with the transfer rods already attached, or the transfer rods may be separately inserted into the bag and attached after the IOL is in the biocompatible bag. The transfer rods may be threaded through transfer rod holes 240a and 240b that were made prior to implantation of the biocompatible bag in the eye or formed after implantation. Attachment of the transfer rods to the zonules or ciliary body can be made as described above.

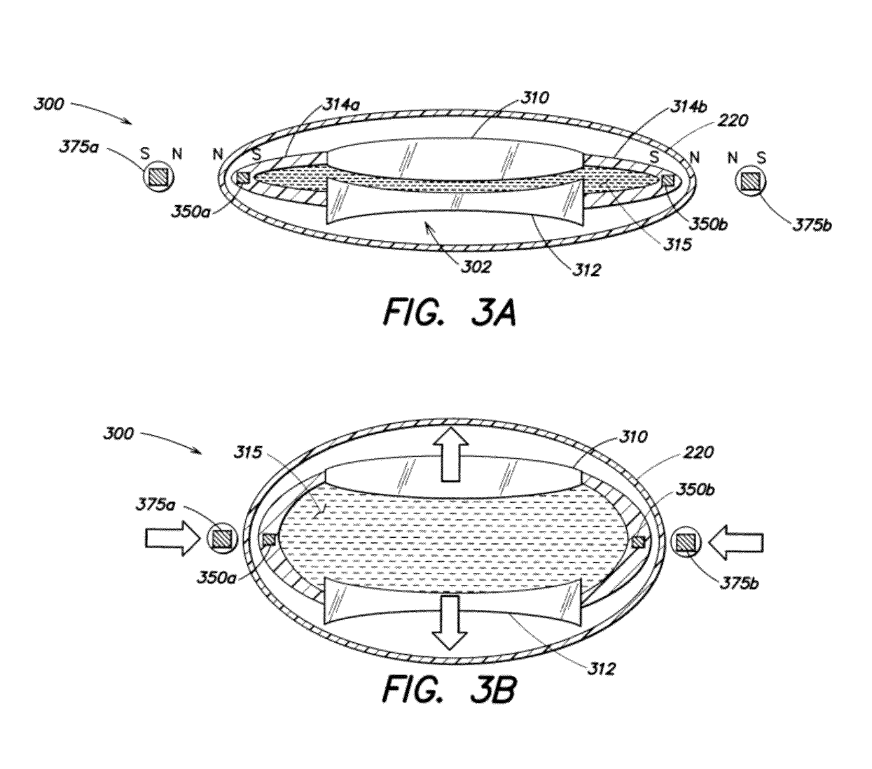

FIGS. 3A and 3B are cross-sectional side views of another embodiment 300 of an accommodating intraocular lens kit for use in embodiments according to aspects of the present invention. Lens 302 comprises two optical elements 310 and 312. In this embodiment, transfer rods are omitted and magnetic media 350a, 350b, 375a and 375b are included for transferring accommodative force of said eye from the eye to the at least one biasing element. The magnetic media comprise magnetic media 375a and 375b that are sized and shaped for coupling to the zonules or ciliary body, and magnetic media 350a and 350b connected to biasing elements 314a and 314b of the lens. Although the illustrated embodiment comprises two magnetic media each one attached to a corresponding one of two biasing elements, it will be appreciated an embodiment of a lens may have one or more biasing elements, each biasing element having one or more magnetic media attached thereto. The space 315 between optical elements 310 and 312 maybe enclosed by the lens and filled with liquid or air or may be open so as to permit the eye's fluid to enter.

After implantation, the magnetic media 350a and 375a, and magnetic media 350b and 375b are aligned such that a magnetic medium in the zonules and a magnet medium in a biasing element have a common pole facing one another. Accordingly, as the ciliary muscle relaxes to achieve distance vision, the magnetic medium in the zonules moves outward permitting radially outward movement of the biasing element as a result of the resiliency of the lens, thereby drawing lenses together; and as the ciliary muscle contracts to achieve near vision, the magnetic medium 375a in the zonules moves radially inward causing the magnetic medium 350a in the biasing element to move radially inward, thereby moving lenses apart from one another. It will be appreciated that, in the present embodiment, unlike the embodiment of FIG. 2A, physical contact between the portion of the means for transferring accommodative force that is connected to the zonules (or ciliary body) and the biasing elements is not necessary. Although magnetic media 375a and 375b were discussed as being connected to the zonules, it should be appreciated that they may be coupled (e.g., using an adhesive) to or rest against the ciliary body.

The magnetic media 350a and 350b may be attached to the biasing elements using any suitable attachment technique that is capable of facilitating transfer of a force from the zonules (or ciliary body) to the biasing elements to move either or both of optical elements 214 and 216 (in the case of a dual optical element lens) and appropriately translate and/or deform an optical element (in the case of a single optical element lens). For example, an adhesive, a clip, pin, snap or suturing may be used to achieve the attachment. To attach the magnetic medium to the zonules, the magnetic medium may be entwined in the zonules, sutured thereto or connected by any other suitable attachment technique.

To implant the components of a kit according to this embodiment, a patient's crystalline lens is first removed using any suitable technique. After the lens is removed, an AIOL including magnetic media connected to the biasing elements can be inserted into the eye using a technique as described above. It will be appreciated that, in the embodiment illustrated in FIGS. 3A and 3B, neither transfer rods nor holes to accommodate transfer rods need not be present. Attachment of the magnetic media to the zonules can be made as described above.

Having thus described the inventive concepts and a number of exemplary embodiments, it will be apparent to those skilled in the art that the invention may be implemented in various ways, and that modifications and improvements will readily occur to such persons. Thus, the embodiments are not intended to be limiting and presented by way of example only. The invention is limited only as required by the following claims and equivalents thereto.

1. An accommodating intraocular lens kit for implantation into an eye, comprising:
an accommodating intraocular lens having at least one biasing element;
a biocompatible bag having a size and shape to conform to an interior surface of a capsular bag; and
a means for transferring accommodative force of the eye from the eye to the at least one biasing element.

2. The kit of claim 1, wherein the accommodating intraocular lens comprises a first optical element and a second optical element.

3. The kit of claim 1, wherein the accommodating intraocular lens consists of a single optical element.

4. The kit of claim 1, wherein the bag has a spring constant of at least 0.5 mN/mm.

5. The kit of claim 5, wherein the bag has a spring constant of at least 1.0 mN/mm.

6. The kit of claim 4, wherein the bag has a spring constant less than 200 mN/mm.

7. The kit of claim 1, where the bag has a hole sized and shaped to permit the accommodating intraocular lens to fit therethrough.

8. The kit of claim 1, wherein the means for transferring comprises at least two transfer rods.

9. The kit of claim 8, wherein the transfer rods each have a length to permit coupling between the biasing elements and the zonules or ciliary body of the eye.

10. The kit of claim 9, wherein the transfer rods each have a length between 2-7 mm.

11. The kit of claim 1, wherein the means for transferring comprises at least one magnetic medium connected to the biasing element and at least one magnetic medium adapted to be connected to the zonules or ciliary body.

12. The kit of claim 1, wherein the means for transferring comprises at one magnetic medium connected to the at least one biasing element.

13. A method of implantation of an accommodative lens in an eye, comprising:
inserting a biocompatible bag into an eye;
inserting an accommodating intraocular lens having at least one biasing element into the biocompatible bag after the biocompatible bag is in the eye; and
connecting a means for transferring accommodative force between the zonules or ciliary body of the eye, and the at least one biasing element.

14. The method of claim 13, wherein the step of connecting the means for transferring comprises connecting a transfer rod between the biasing element and the zonules or the ciliary body of the eye.

15. The method of claim 13, wherein the step of connecting the means for transferring comprises connecting at least one magnetic medium to the at least one biasing element
and connecting at least one magnetic medium to the zonules or ciliary body.
